(19) United States
(12) Patent Application Publication
(10) Pub. No.: US 2019/0167152 A1
(43) Pub. Date: Jun. 6, 2019

(54) METHOD AND APPARATUS FOR THE DIAGNOSIS OF PNEUMONIA USING EXHALED BREATH METABOLOMICS

(71) Applicant: KONINKLIJKE PHILIPS N.V., EINDHOVEN (NL)

(72) Inventors: JOHANNES WEDA, NIJMEGEN (NL); HUGO KNOBEL, EINDHOVEN (NL); TAMARA MATHEA ELISABETH NIJSEN, WEERT (NL); SANNE DE BRUIN, AMSTELVEEN (NL); POULINE van OORT, AMSTERDAM (NL); MARCUS J. SCHULTZ, Abcoude (NL); LIEUWE DURK JACOBUS BOS, AMSTERDAM (NL)

(21) Appl. No.: 15/828,647

(22) Filed: Dec. 1, 2017

(51) Int. Cl. A61B 5/083; G01N 33/497; G01N 33/00; G16H 50/20

(52) U.S. Cl. CPC A61B 5/083; G01N 33/497; G01N 33/0047; G01N 2800/12; G01N 2033/4977; G01N 2033/4975; G16H 50/20

(57) ABSTRACT

A method and apparatus for the diagnosis of hospital-acquired pneumonia is described. The method uses the analysis of volatile organic compounds (VOCs) in exhaled breath that indicate pneumonia or the presence of pathogens in the respiratory tract in intubated and mechanically ventilated intensive care unit patients. The apparatus may be an electronic nose incorporated into a ventilation system, which outputs to a display the indication of pneumonia. One particular useful VOC is 1-propanol.

### BACKGROUND OF THE INVENTION

Severe community- and hospital-acquired pneumonia (CAP and HAP) represent a major clinical problem associated with a high mortality and frequently requires admission to the intensive care unit (ICU), intubation and mechanical ventilation. The diagnosis of CAP and HAP is currently based on clinical, radiological and microbiological criteria, but these criteria have several disadvantages. Physical examination has a high inter-observer variability and a moderate sensitivity and specificity. Chest X-ray has a poor sensitivity and positive predictive value for CAP and HAP. Bacterial cultures need several days before showing growth and results could be false-negative due to previously administered antibiotics. These problems delay the start of targeted therapy.

During this delay time, patients receive empirical broad-spectrum antibiotics that increase the likelihood of the occurrence of multi-drug resistant microorganisms. In addition, there is no method currently available to monitor the antibiotics treatment. Therefore antibiotics are described for a longer period of time, which increases the likelihood of the development of antibiotics resistance.

In addition, to diagnose VAP and also to provide information on targeted antibiotics therapy a bronchoscopy is necessary to obtain a sample. This procedure may be difficult or harmful to certain patients receiving mechanical ventilation.

What is needed therefore is an objective, non-invasive bedside test, which ideally enables rapid exclusion of the presence of pneumonia and/or identifies the causative pathogen. A test which also reduces the need for bronchoscopy-obtained samples would also be beneficial

Exhaled breath contains metabolites in the gas phase called volatile organic compounds (VOCs) that are produced by the host and bacteria. Different bacterial strains show distinct patterns of VOCs in vitro and in animal models. Therefore exhaled breath analysis might be used to identify the causative pathogen in patients suspected of CAP/HAP. A recent study shows that exhaled breath analysis can discriminate between VOC profiles of patients with a high risk of developing nosocomial pneumonia with and without a significant pathogen load in the lower respiratory tract. Thermal desorption with gas chromatography coupled to mass spectrometry (TD-GC-MS) may be used to separate, quantify and identify VOCs.

### SUMMARY OF THE INVENTION

The inventors have discovered a method for determining which VOCs could be used to identify patients with CAP or HAP using GC-MS. The discovery indicates that VOCs in exhaled breath can be used to discriminate between intubated and mechanically ventilated patients on ICU with CAP or HAP and ventilated patients without pneumonia with accuracy. 1-pronanol in particular was found to be consistently lower in patients with pneumonia and, independently, also in patients with colonized airways and might be a marker for bacterial presence and growth. This finding was unexpected and non-intuitive, because it had been assumed that VOC levels in exhaled breath would generally be higher in patients with pneumonia and/or colonized airways.

An invention arising from the discovery is a method for analyzing a patient's breath to detect disease such as pneumonia, comprising the steps of providing a breath detector apparatus operable to capture and hold a volatile organic compound (VOCs) that is contained within a exhaled breath, providing a breath VOC analyzer in communication with the breath detector apparatus, the breath VOC analyzer operable to automatically determine a level of the VOC. The method continues by capturing the VOC with the breath detector apparatus, analyzing the VOC to automatically determine the level, and by comparing the level of the VOC to a predetermined threshold level of the VOC. The method outputs an indication of disease if the level of the VOC is less than the threshold level of the VOC.

The VOC of particular preference is 1-propanol. Preferably the breath VOC analyzer may monitor a patient's exhaled breath for disease in several ways. The analyzer may monitor continuously, in for example, an electronic nose arrangement. The analyzer may alternatively monitor via a regular spot check. The breath VOC analyzer may be incorporated into the patient circuit of a medical ventilator. Alternatively, the VOC analyzer may be a separate TD-GC-MS device, where the VOC of interest is captured by a sorbent tube or by a bag. The breath may be alternatively be captured by a solid phase micro-extraction (SPME) needle, a needle trap, or the like. Depending on the threshold and expected VOC concentration, a direct GC method of measurement might be employed.

In accordance with another aspect of the invention, an apparatus for analyzing exhaled breath to detect pneumonia in a patient is described. The apparatus comprises a breath detector apparatus operable to capture and hold a volatile organic compound (VOCs) that is contained within a exhaled breath, and a breath VOC analyzer in communication with the breath detector apparatus, the breath VOC analyzer operable to automatically determine a level of the VOC. The apparatus includes a hardware computer processor configured to compare the level of the VOC to a predetermined threshold level of the VOC, to output a disease signal if the level of the VOC is less than the threshold level of the VOC. The apparatus alerts the user to a diagnostic condition by use of a display in communication with the hardware computer processor, the display operable to provide an aural or visual alert of the disease indication.

Like the method described previously, a preferable VOC is 1-propanol. A preferable breath VOC analyzer is an alternative as described above. The device may be incorporated with a medical ventilator. An alternate analyzer is a TD-GC-MS analyzer, where the VOC of interest is captured by a sorbent tube, a bag, an SPME needle or the like.

### BRIEF DESCRIPTION OF THE FIGURES

FIG. 1 illustrates a flowchart of screened patients, in accordance with the present invention.

FIG. 2 illustrates plots of an ion count of VOCs in 4 groups (control, patients with colonization, patients with possible pneumonia, and patients with probable pneumonia from left to right) that showed a p-value <0.05 between patients with a probable pneumonia compared to controls.

FIG. 3 illustrates a volcano plot comparing patients with probable/proven pneumonia versus control populations.

FIG. 4 illustrates a first (PC1) and second (PC2) principal component analysis of the method discovery.

FIG. 5 illustrates a plot of ion counts for VOCs that show a p-value <0.001.

FIG. 6 illustrates a volcano plot comparing patients with probable/proven pneumonia versus control populations.

FIG. 7 illustrates one preferred embodiment of the inventive apparatus.

FIG. 8 illustrates a flow chart of one embodiment of the inventive method.

### DETAILED DESCRIPTION OF THE INVENTIVE CONCEPTS

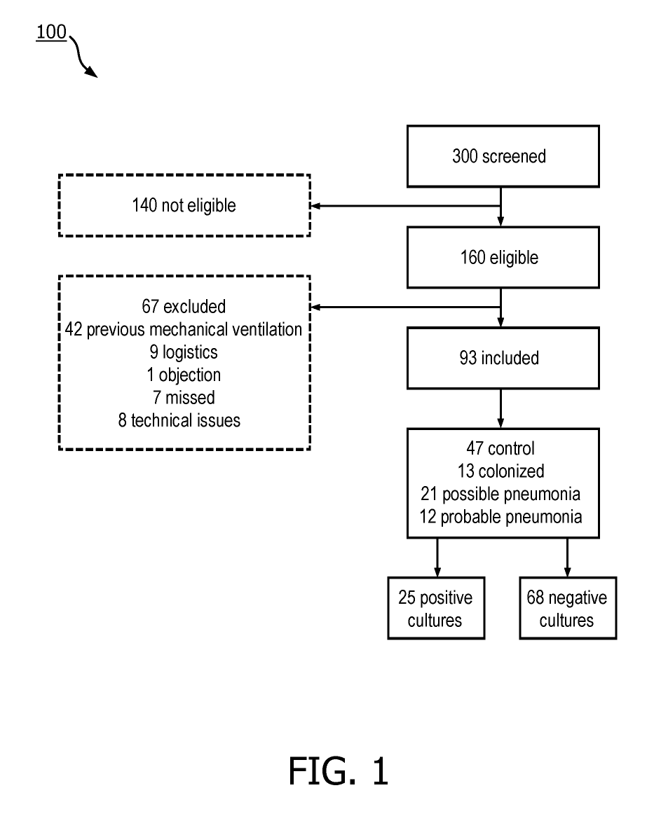

FIGS. 1 through 6 may be viewed in correspondence with the following description of how the invention was discovered. FIG. 1 illustrates a chart 100 of a study population, wherein a total of 300 patients were screened, of whom 160 were eligible. Sixty seven (67) patients were excluded for several reasons e.g. previous mechanical ventilation or technical issues. Ninety three (93) patients were thus included. Twelve patients (13%) had probable pneumonia and were considered cases. 47 patients (50%) were not suspected of pneumonia and did not have colonized airways and were included as controls. 21 patients had a possible pneumonia (23%), and 13 patients who were not suspected of pneumonia but had colonized airways. In total, 25 (27%) patients had colonized airways, irrespective of the suspicion of pneumonia. The baseline demographic and clinical characteristics of the study population are shown in Table 1 below.

TABLE 1
Patients demographics and clinical characteristics, data are presented as median (interquartile range) or n (%)

| | | | Possible | | Probable | | | | |
|---|---|---|---|---|---|---|---|---|---|
| | Control | | Colonization | | Pneumonia | | Pneumonia | | P- |
| | N = 47 | | N = 13 | | N = 21 | | N = 12 | | value |
| Age at ICU admission | 59 | (48-70) | 64 | (43-79) | 63 | (55-71) | 61 | (45-72) | 0.93 |
| Patient gender | | | | | | | | | |
| Female | 16 | (34) | 5 | (38) | 6 | (29) | 7 | (58) | 0.41 |
| Male | 28 | (59) | 8 | (62) | 15 | (71) | 5 | (42) | |
| Admission type | | | | | | | | | |
| Medical | 31 | (65) | 8 | (62) | 20 | (95) | 11 | (92) | 0.17 |
| Surgical elective | 1 | (2) | 0 | (0) | 0 | (0) | 0 | (0) | |
| Surgical emergency | 12 | (25) | 5 | (38) | 1 | (5) | 1 | (8) | |
| ICU Length of stay | 3 | (2-5) | 3 | (2-4) | 4 | (3-5) | 5.5 | (3-9) | 0.18 |
| (days) | | | | | | | | | |
| APACHE IV Score | 80 | (55-97) | 76 | (56-89) | 76.5 | (57-103) | 66 | (59-83) | 0.74 |
| ICU mortality | 11 | (23) | 1 | (8) | 2 | (10) | 4 | (33) | 0.20 |
| ARDS | 2 | (4) | 12 | (92) | 15 | (71) | 9 | (75) | <0.001 |
| Positive Cultures | 0 | (0) | 13 | (100) | 3 | (14) | 9 | (75) | <0.001 |
| Comorbidity | | | | | | | | | |
| Malignancy | 4 | (9) | 3 | (23) | 4 | (19) | 4 | (33) | 0.18 |
| Diabetes Mellitus type 2 | 4 | (9) | 3 | (23) | 2 | (10) | 2 | (17) | 0.55 |
| COPD | 1 | (2) | 0 | (0) | 4 | (19) | 1 | (8) | 0.054 |
| Asthma | 0 | (0) | 0 | (0) | 1 | (5) | 0 | (0) | 0.49 |
| Other | 1 | (2) | 0 | (0) | 1 | (5) | 1 | (8) | 0.72 |
| Pmax cm H₂O | 17 | (14-22) | 16 | (13-17) | 21 | (18-24) | 25 | (22-28) | 0.004 |
| Peep cm H₂O | 5 | (5-5) | 5 | (5-5) | 8 | (5-10) | 9.5 | (5-10) | 0.001 |
| Tidal Volume mL | 458 | (391-525) | 467 | (448-581) | 500 | (383-576) | 464 | (409-575) | 0.74 |
| FiO₂ % | 40 | (35-40) | 35 | (35-40) | 40 | (35-45) | 45 | (40-60) | 0.024 |
| PaO₂ kPa | 13.8 | (12..2-17) | 16.3 | (13.7-24.) | 14.7 | (12.4-17.7) | 14.2 | (10.9-19.0) | 0.31 |
| PaCO₂ kPa | 5.1 | (4.5-5.6) | 5.1 | (4.6-5.4) | 5.5 | (4.7-5.7) | 5.1 | (4.5-6.1) | 0.58 |

Determination of Probable Pneumonia Vs. Patients without Pneumonia and without Colonized Airways

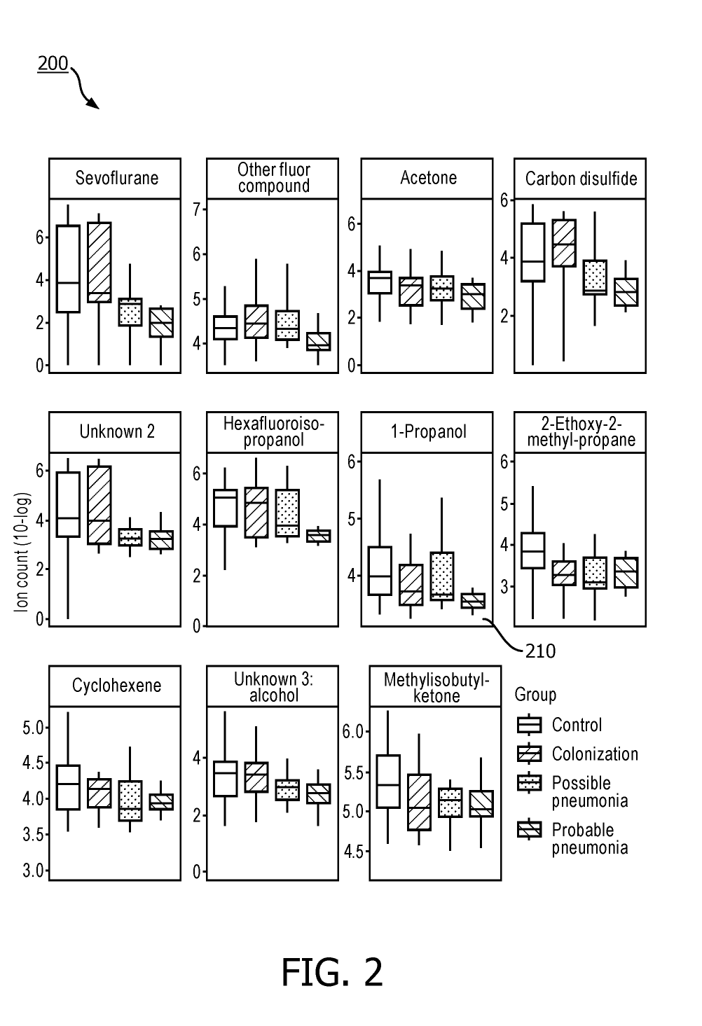

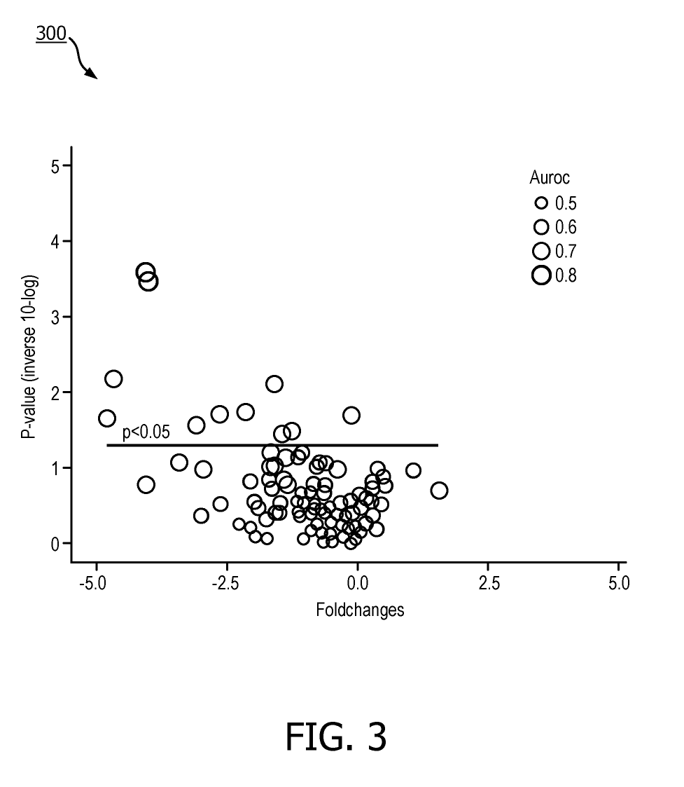

145 VOCs were found in the breath of all patients. Eleven (7.6%) VOCs were significantly lower in the breath of cases than in that of controls (p-value <0.05). FIG. 2 illustrates a plot 200 of the distribution and names of these VOCs of interest ion counts of VOCs in 4 groups (control, patients with colonization, patients with possible pneumonia, and patients with probable pneumonia from left to right) that showed a p-value <0.05 between patients with a probable pneumonia compared to controls. The results of this listing may be visualized in a volcano plot 300, as shown in FIG. 3. The FIG. 3 volcano plot 300 compares patients with probable/proven pneumonia vs. controls. Each dot represents a VOC. The y-axis shows the inverse of the 10-log transformed p-value: the higher on the axis, the more significant. The x-axis shows the fold change between the groups. The size of the dots represents the AUROC.

Ten out of these VOCs of interest showed an “area under the receiver operating characteristics curve” (AUROC) higher than 0.7. 1-Propanol at 210 and hexafluoroisopropanol showed the highest AUROC of respectively 0.83 (CI 0.72-0.93) and 0.82 (CI 0.72-0.93). 1000 permutations of the labels were performed and 1.7% and 2.3% of these random scenarios resulted in a similar or better p-value and AUROC, respectively.

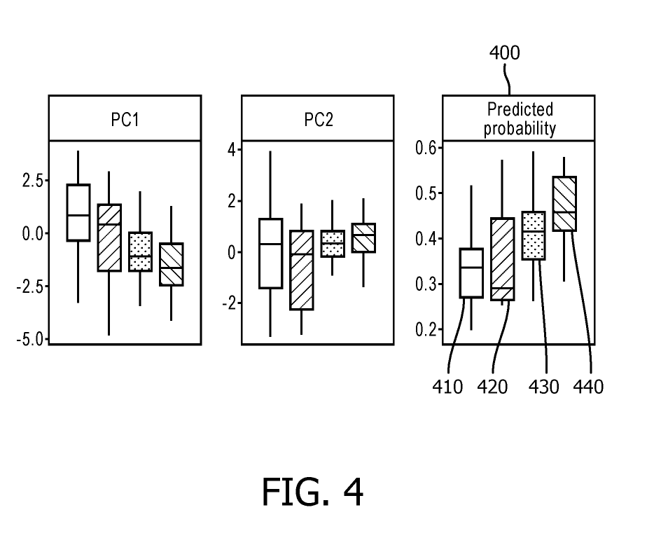

Principal component analysis was found to show a significant lower first principal component score (explaining 35.1% of variance) for patients with probably pneumonia (p<0.001). FIG. 4 illustrates a first (PC1) and second (PC2) principal component explained 35.1% and 22.4% of the variance, respectively. Predicted probability 400 is calculated by the PLSDA model. From left to right appear the results for controls 410, colonized controls 420, possible pneumonia 430 and probable pneumonia 440. The second principle component (22.4% of variance) did not show significant differences (p=0.43) between cases and controls.

Partial least squares discriminant analysis (PLSDA) was used to classify cases and controls, as shown in Table 2 below. The AUROC for the PLSDA model was 0.87 [95%-CI: 0.75-0.98] for in-set analysis and 0.73 [95%-CI: 0.57-0.88] after leave-one-out cross-validation. Prediction of pneumonia probability in patients with possible pneumonia and without pneumonia with colonized airways results gave results in between cases and controls. FIG. 4 illustrates.

TABLE 2
2 × 2 tables. PLSDA model was trained with significant VOCs.

| | | Probable | |
|---|---|---|---|
| | | pneumonia | Control |
| In-set analysis | Probable pneumonia | 5 | 3 |
| | Control | 7 | 44 |
| Leave-one-out validation | Probable pneumonia | 3 | 4 |
| | Control | 9 | 43 |
| | | Positive | Negative |
| | | culture | culture |
| In-set analysis | Positive culture | 7 | 5 |
| | Negative culture | 18 | 63 |
| Leave-one-out validation | Positive culture | 5 | 6 |
| | Negative culture | 20 | 62 |

Patients with Colonized Airways Vs. Patients without Colonized Airways

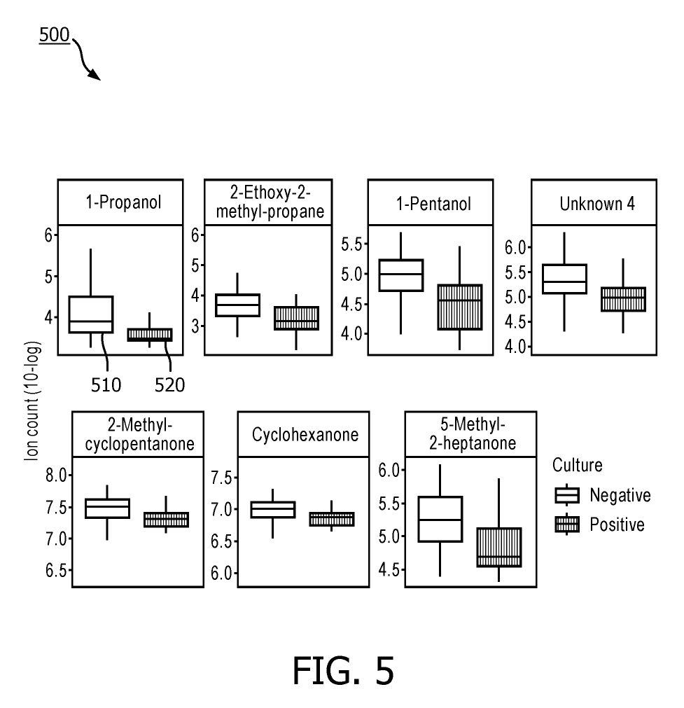

Fifty two (52) VOCs (35.9%) were found to be significantly lower in patients with colonized airways than in patients without colonization (p-value <0.05). FIG. 5 illustrates a plot 500 of the results, where seven of these VOCs showed a p-value <0.001. Moreover, 11 out of the 52 VOCs showed an AUROC of above 0.7. Of particular note is the 1-propanol VOC result, comparing patients without colonization 510 to patients with colonization 520.

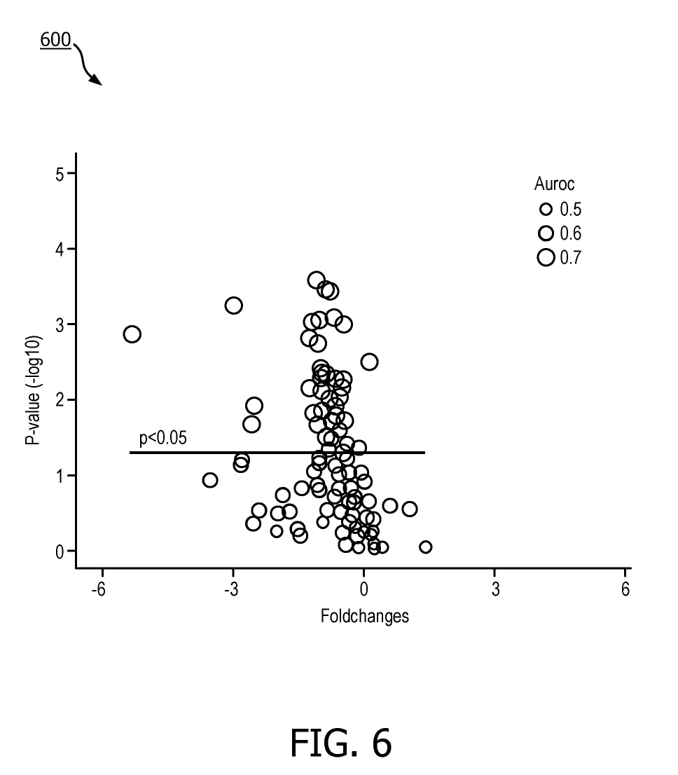

These results may also be visualized in a volcano plot at FIG. 6. 1000 permutations of the labels were performed and 1.4% and 0.06% of these random scenarios resulted in a similar or better p-value and AUROC, respectively. The volcano plot 600 compares patients with probable/proven pneumonia vs. controls. Each dot represents a VOC. The y-axis shows the inverse of the 10-log transformed p-value: the higher on the axis, the more significant. The x-axis shows the fold change between the groups. The size of the dots represents the AUROC. The horizontal line shows p=0.05 with dots above this line having p<0.05.

Principal component analysis indicates a significantly higher first principal component score (explaining 62.5% of variance) for patients with colonized airways (p<0.01). The AUROC for the PLSDA model was 0.79 [95%-CI: 0.70-0.90] for in-set analysis and 0.69 [95%-CI: 0.57-0.82] after leave-one-out cross-validation.

### DISCUSSION

The results of the study indicate that intubated and mechanically ventilated ICU patients with and without pneumonia can be discriminated with moderate to good accuracy with exhaled breath analysis by GC-MS. Patients with colonized airways or with a low suspicion of pneumonia were classified as an intermediate group, in between pneumonia and control. Airway colonization, irrespective of the likelihood of pneumonia, also resulted in a changed concentration of several VOCs in the exhaled breath.

The inventors discovered a moderate to good accuracy with their models after leave-one-out cross-validation, but several other studies on breath analysis in pneumonia have reported higher diagnostic accuracies. Schnabel et al., for example, reported an AUROC of 0.87 in diagnosing ventilator associated pneumonia (VAP). All included patients in that study underwent a diagnostic broncho-alveolar lavage. Although the optimal diagnostic strategy for pneumonia is discussed there, broncho-alveolar lavage is generally considered a better gold standard and this may partly explain the higher accuracy that was found previously.

The inventors found eleven VOCs, see FIG. 2, that were considered significant when distinguishing between patients with a probable pneumonia and controls (p<0.05). Sevoflurane, hexafluoroisopropanol and the other fluor compound were probably of an exogenous origin and could thus be regarded as falsely discovered. Acetone is generally present in high concentrations in breath and is also produced by most bacteria. Carbon disulfide is a volatile liquid that is frequently used as a chemical or industrial solvent. 1-Propanol is most importantly produced by E. coli, which might use this alcohol to hinder growth of other pathogens. Some propanes are used as fuels (e.g. for engines or residential central heating) and might thus be of false-discovery as well. Cyclohexene is a hydrocarbon that is used to fabricate other chemicals. The production of methyl ketones occurs during decarboxylation of fatty acid derivates and the longer 2-ketones have been described as classical biomarkers for P. aeruginosa.

All discriminative molecules were found in lower concentrations in patients with pneumonia compared to controls, as well as in colonized patients compared to non-colonized patients. This is a remarkable finding since the prior art expected that most biomarkers increase during pneumonia. Furthermore, this result has not previously been reported in breath profiles studies about respiratory tract infections. No other studies about breath profiling are known to have been performed in patients with CAP or HAP. However, several studies have been conducted in patients with other inflammatory pulmonary diseases, including but not limited to asthma, chronic obstructive pulmonary disease (COPD), Acute Respiratory Distress Syndrome (ARDS) and ventilator-associated pneumonia (VAP). The same trends were observed in the comparison of COPD patients and controls; VOCs that discriminated most, were predominately lower in COPD patients. Care should be used while extrapolating these results from chronic inflammation to acute illness, but such extrapolation suggests that inflammation can lead to a decreased concentration of certain VOCs in exhaled breath. Schnabel et al, for example, also found some VOCs that were decreased in patients with VAP. Nevertheless, more than half of the discriminative VOCs were higher in patients with VAP compared to controls. The cause of decreased VOCs is yet unclear. The inventors hypothesize that inflammation caused by pneumonia could lead to altered gas exchange over the lung-blood barrier, resulting in decreased VOC excretion. Alternatively, inflammatory or bacterial cells may use the VOCs or their metabolic precursor, resulting in a lower concentration in the exhaled breath. Furthermore, infection or colonization could alter the normal microbiome in the lower and upper respiratory tract due to inflammation, overgrowth of certain pathogens or administration of antibiotics. The decreased VOCs could reflect the suppression of the lung microbiome. Finally, one of the reasons that this study did not confirm that specific VOCs produced by bacteria increase during infection, could be that the significant changes found in this study were all part of the host response and less influenced by breath profiles from bacteria. Twelve patients were diagnosed with a probable pneumonia. For these patients the inventors found nine different pathogens. In other words, the frequency of each pathogen across the pneumonia group is too low. Each pathogen produces its own specific breath profile. Due to the low frequency of each pathogen the inventors may have missed the statistical power to find significant VOC compositions produced by the bacteria, within the pneumonia group.

We found that the VOCs that discriminated between patients with pneumonia and controls and between colonized and non-colonized airways were different ones; only six out of 57 VOCs matched. 1-Propanol was the only VOC that was highly discriminatory in both analyses. Therefore, this is the only VOC identified in this study that might qualify as a biomarker. Remarkably, more VOCs were significantly different between patients with and without colonized airways and the amount was higher than the amount of VOCs that distinguished pneumonia from controls. Furthermore the majority of the relevant VOCs related to pneumonia had an AUROC above 0.7, while the majority of the VOCs related to finding the colonization status had an AUROC of less than 0.7. Thus the significantly altered VOCs related to pneumonia were stronger predictors. VOC formation and depletion have a complicated balance. The relative composition of VOCs in exhaled air can change as a result of a disease that may lead to a decrease or an increase of a certain compound. VOCs could be produced by the host or by the bacteria. The inventors hypothesize that in patients with a colonized respiratory tract the signal is predominantly altered by the bacteria, while in investigating pneumonia the signal is also influenced by host-response. That these two processes contribute to changes in exhaled breath VOCs has been nicely demonstrated in animal studies.

The predicted probability for having pneumonia for patients that had colonized airways without pneumonia or had a possible pneumonia were in between the values that were found for the control group and patients with a probable pneumonia. This result was expected, because controls and patients with probable pneumonia represented the extremes in the spectrum of pneumonia, the remaining patients exemplified as subjects lying somewhere in between these two extremes. This finding emphasizes the plausibility of the used model.

The exhaled breath samples that were used by the inventors were a mixture of alveolar and dead space air. This methodology was chosen because it represents a safe, non-invasive method that is easy to perform. Breath was collected in tubes, which for example were connected at a sample rate of 200 milliliters/minute for ten minutes to the circulation circuit, leading to a sampled volume of two liters. However, with a dedicated method for detecting e.g. 1-propanol, lower sampling volumes could be an option. It was assumed that this is sufficient to collect most VOCs in exhaled breath. Furthermore, in the control group significantly less patients were diagnosed with ARDS. Previous studies showed that ARDS results in altered breath profiles. It is unclear how the unequal distribution of patients with ARDS influenced the results of this study, although it should be noted that none of the identified VOCs were predictive of ARDS in a previous study. One of the strengths of this study is that it did not only compare patients with pneumonia to controls but it also compared colonized and non-colonized patients. There is a clinical relevant difference between merely the presence of bacteria versus the presence of bacteria that actually leads to infection. The inventors were able to see that different VOCs discriminate between these conditions.

Another strength of this study is in the group selection the inventors used for building the classification model for predicting the probability for pneumonia. Only patients with a high suspicion or without any suspicion for respiratory tract infections were used to train the algorithm. Because of the lack of a good gold standard, two clinically well-defined groups were needed to determine reliably the accuracy of this new diagnostic test. Another strength is that the accuracy of the model was assessed with the AUROC as measure of accuracy that is proven suitable in classifying patients.

GC-MS analysis may be relatively impractical as a method for VOC detection in clinical practice. Specialized personnel are required, or may not be available at the bedside, and the analysis is time-consuming. However, GC-MS is currently considered the gold standard for identifying distinct VOCs. An electronic e-nose may be preferable because it is faster and recognizes patterns, but cannot currently distinguish specific VOCs. The inventors contemplate a sensor array (electronic nose or eNose) that can rapidly detect the described VOCs to accurately diagnose or exclude pneumonia, and that can be developed using existing technology. Alternatively, if one knows the VOC of interest beforehand, GC or uGC with other detectors (being much simpler to use) could be used. Also techniques like IMS (ion mobility spectrometry) could be used as an alternative to GCMS or eNose. Using an e-nose is preferable because it is non-invasive, fast and completely safe. This and future studies can be used as a reference for which VOCs should be targeted with selective sensors.

In the above study, exhaled breath was sampled and analyzed by standardized and existing methodology. Breath was collected through a disposable side-stream connection for 10 minutes and VOCs were stored on a sorbent tube. These tubes were analyzed by means of thermal desorption GC-MS. Ion-fragments were detected and retention time correction was performed with a known statistical analysis package. Ion counts of fragments within a small window of retention times (+/−3 seconds) were summed to get a total ion count if they strongly correlated (loaded onto the same principal component) in order to limit collinearity of the predictor matrix (e.g. to get one intensity per patient per VOC) but still allow for differentiation between co-elutions.

Embodiments of the Inventive Apparatus

Several embodiments of the inventive apparatus adopt the discoveries and methods as described above. Each embodiment includes at least four elements. First is a breath detector apparatus that is operable to capture and hold a VOC that is contained within a volume of exhaled breath or gas. Connected to the breath detector apparatus is a breath VOC analyzer which is operable to determine a level or concentration of the captured VOC. A hardware computer processor may control the analyzer and in some embodiments the breath detector. The hardware computer processor is further configured to compare the determined level of the VOC to a predetermined threshold level of that VOC. The hardware computer processor provides an output signal indicating the presence of disease if the level of the VOC is less than the threshold level of the VOC. Fourth, a display or a user interface receives the processor output signal and provides an aural or visual alert of the disease indication.

The apparatus preferably discriminates the VOC 1-propanol, which as described above, indicates a pneumonia condition if the level is below a threshold level for the VOC, such as an ion count (10-log) of 3.7.

Following are particular embodiments of the inventive apparatus.

### Embodiment 1

An on- or offline system samples and analyses breath and uses the profile of 1-pronanol to give decision support in the context of diagnosis and treatment monitoring for hospital and community acquired pneumonia. The decision support can contribute to the diagnosis, give guidance for further diagnosis methods to use and can contribute to antibiotics stewardship.

### Embodiment 2

An on- or offline system that samples and analyses breath and uses the profile of multiple VOCs mentioned in FIG. 2 to give decision support in the context of diagnosis and treatment monitoring for hospital and community acquired pneumonia

One offline system includes a breath detector apparatus operable for taking breath samples by means of a patient exhaling into a bag (e.g. a Tedlar bag or another storage material, or a sampling apparatus containing a storage material) for a specified time. Afterwards a pump and a mass flow controller are connected to the bag and the collected air is pushed or pulled with a fixed flow for a fixed amount of time through a sorbent tube.

Some mechanically ventilated patients however may not be able to breathe into a bag. An alternate embodiment of this arrangement may be by means of a small pump at the bed side which pulls breath samples directly from the patient exhalation gas through the sorbent tube.

After collecting the VOC of interest in the breath detector apparatus, the gas samples may be analyzed using gold standard chemical analytical techniques such as Gas Chromatography Mass-Spectrometry (GC-MS), Time Of Flight Mass Spectrometry (TOF-MS) and Ion-Mobility Spectrometry (IMS). These known techniques provide knowledge on individual molecular compounds and can provide precise measures on the marker abundance in the breath samples. These methods, however, require rather laborious procedures, relatively large devices and trained operators.

In an embodiment involving an online approach, the exhaled breath is passively or actively transported to a sensor or an array of sensors. For monitoring this approach has a strong preference due to the ease and speed of processing. Using this method the breath analyzer can be embedded in the device that samples the patient's exhaled breath as drawn from the ventilator hoses (i.e. using a pump).

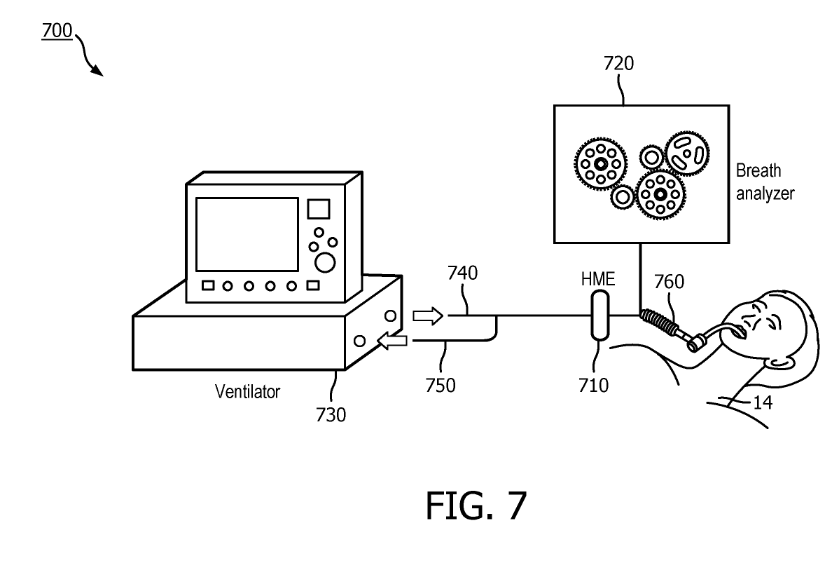

FIG. 7 illustrates a schematic overview of such a system 700 for analyzing exhaled breath, which may optionally be disposed in conjunction with a medical ventilator 730 which provides a patient circuit that provides gas through an inhalation tube 740 and exhausts patient expiratory gases through an exhalation tube 750. In this embodiment, the exhalation port 760 in the patient circuit, shown here intubated, is the source of the exhaled breath from a patient 14. A side-stream “draw” from port 760 provides a continuous sample flow of patient breath to a combined breath detector and breath VOC analyzer 720. Detector/Analyzer 720 includes a VOC capture mechanism and analyzing mechanism under control of the hardware computer processor. Detector/Analyzer 720 preferably includes an output display as well, for providing aural and visual indication of disease such as the indication of pneumonia.

An Electronic Nose (eNose) can optionally provide the on-line analysis needed at analyzer 720 to detect the VOC of interest. An eNose consists of an array of non-specific gas chemical sensors combined with a chemometric processing tool. Different known techniques exist for the precise type of chemical sensor and chemometric processing methods. The choice of sensor and processing method may be based upon the distinguishing biomarker. Options include molecular imprinting or optical techniques using infrared lighting. Adjusting the threshold while balancing sensitivity and specificity, an optimal setting can be found by normal experimentation by one of ordinary skill in the art. As shown, the eNose can be disposed to directly sample exhaled breath from the exhalation tube or port 760.

A lower end miniaturized GC, a so-called μGC, can also be used to detect these volatiles of interest. These μGCs form a promising technique for bedside usage. In these devices the full gas chromatography (basically comprising 4 components: a pump, a pre-concentrator, a separation column and a detector) may be implemented on a chip. The main function, i.e. separation, is realized by the micro column. The detector function of the μGC can be implemented with suitable small gas detectors, such as piezoelectric cantilevers, MOx layers, photo-ionisation detectors, or thermal conductivity detectors.

### Embodiment 3

In yet another alternative embodiment, the breath analyzer is embedded in the ventilator system 730 itself. Such an arrangement obviates the need for an additional device at the patient's bedside, and allows continuous monitoring of breath. For embedding the breath analysis into the ventilator system, eNose type of techniques can also be used. Care should be taken not to interfere with the ventilator in terms of pressures and flows, also in the context of regulatory issues. Therefore, a side stream approach as mentioned above is preferred. Such a side stream approach can however still be integrated in the ventilator device, avoiding extra devices at the bedside, and avoiding abrupt pressure changes in the ventilator systems, which may harm the vulnerable lungs.

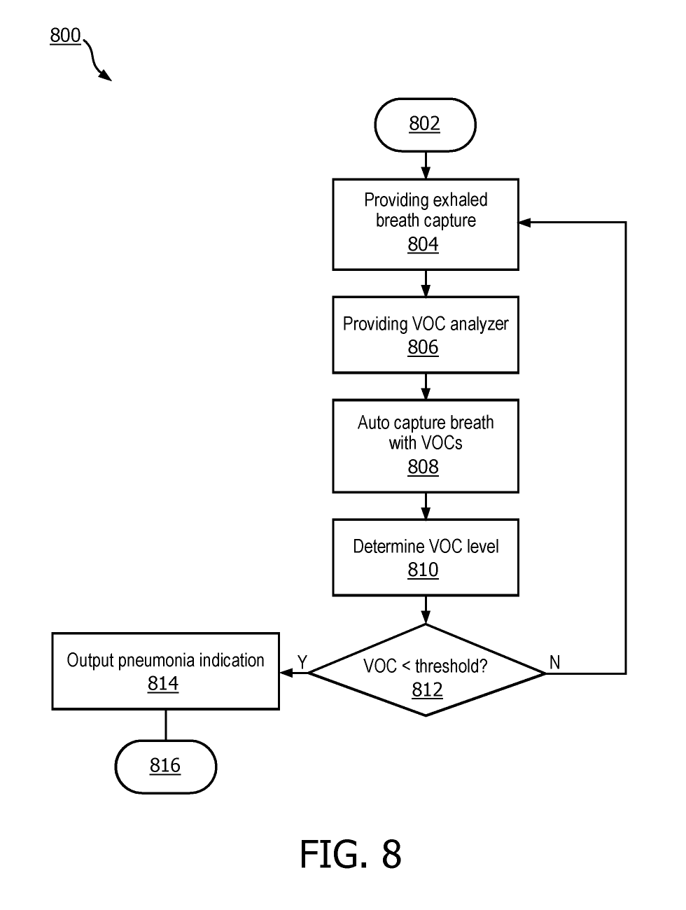

Now turning to the FIG. 8 flow chart, a method 800 is described for analyzing a patient's breath to detect disease. The method starts at step 802 by initiating a breath capture and analyzing apparatus. Step 804 provides one of the breath detector apparatus' embodiments as described above. The detector apparatus is operable to capture and hold a VOC of interest that is contained within an exhaled breath. The detector apparatus may be a sorbent tube through which a specified volume of exhaled gas is drawn by a pump. The exhaled gas may be patient breath comprised of a combination of alveolar air and dead space air, in order to maximize comfort and ease of use.

Also provided at a step 806 is a VOC analyzer, as described above. The VOC analyzer may be a TD-GC-MS device, or may be an electronic nose (eNose) that is arranged to detect a particular VOC of interest. The VOC analyzer is in communication, either on-line or off-line, with the breath detector apparatus. The breath VOC analyzer is operable to automatically determine a level of the VOC.

The preferred VOC of interest for detection of disease is 1-propanol. However, it is envisioned that acceptable results in detecting disease may also be obtained by capturing and analyzing for one or more of the following VOCs: propanes or alkylethers, methyl ketones or other ketones such as 2-ketones, propanols, carbon disulfide, and acetone. Pneumonia is the particular disease of interest for detection using one or more of these VOCs. Capturing step 808 comprises capturing one or more VOCs of interest, e.g. 1-propanol, from the exhalation gas stream with the breath detector apparatus. The capture at step 808 may be conducted continuously via the eNose device, or may be automatically conducted as desired or on a periodic basis under control of a hardware computer processor. A sorbent tube or a retention bag may also be used.

The captured VOC from step 808 is then analyzed at step 810, wherein the concentration or level of the VOC is determined. As previously described, this step may be conducted by means of a TD-GC-MS analyzer, an eNose, or other known methods.

After the level of VOC(s) of interest is determined at step 810, the VOC level is compared at comparing step 812 to a predetermined threshold level for that VOC of interest. One preferred embodiment is a VOC 1-propanol level which is compared to a threshold level. FIG. 2 for example indicates a threshold VOC level of about 0.36 ion count (10-log), but this threshold depends on the analyzing device used. Other manufactures of detectors might use different names to express concentration levels of VOCs, and such level names fall within the scope of the invention. If the 1-propanol level in the sample is less than the threshold level, pneumonia is indicated. In this case, a corresponding signal output to a user interface is then provided to an outputting and indicating step 814 to indicate the possible presence of disease (pneumonia). The disease condition is preferably displayed by visual or audible message, alarm, or alert such that the care provider can respond appropriately. The method then exits at ending step 816.

If the level of VOC is higher than the predetermined threshold, the method may return to the providing step 804 to conduct another test upon a predetermined periodicity, or as commanded by the user.

The invention encompasses modifications to methods and apparatus' that can be integrated into known ventilator systems. The invention may be enabled for example in intensive care units (ICU's), electronic noses, or other dedicated devices targeted to easily diagnose CAP and/or HAP from breath samples.

We claim:

1. A method for analyzing a patient's breath to detect disease, comprising the steps of:
providing a breath detector apparatus operable to capture and hold a volatile organic compound (VOCs) that is contained within a exhaled breath;
providing a breath VOC analyzer in communication with the breath detector apparatus, the breath VOC analyzer operable to automatically determine a level of the VOC;
capturing the VOC with the breath detector apparatus;
analyzing the VOC to automatically determine the level;
comparing the level of the VOC to a predetermined threshold level of the VOC; and
outputting an indication of disease if the level of the VOC is less than the threshold level of the VOC.

2. The method of claim 1, wherein the VOC comprises 1-propanol.

3. The method of claim 2, wherein the predetermined threshold level of the VOC is about 0.36 ion count (10-log).

4. The method of claim 1, wherein the VOC comprises one of an alkylether, a methyl-ketone, a propanol, a carbon disulfide, and an acetone.

5. The method of claim 1, wherein the step of providing a breath analyzer comprises providing an electronic nose operable to detect the VOC.

6. The method of claim 1, wherein the step of providing a breath detector apparatus comprises providing a sorbent tube.

7. The method of claim 6, wherein the exhaled breath comprises a combination of alveolar air and dead space air.

8. The method of claim 1, wherein the disease comprises pneumonia.

9. An apparatus for analyzing exhaled breath to detect pneumonia in a patient, comprising:
a breath detector apparatus operable to capture and hold a volatile organic compound (VOCs) that is contained within a exhaled breath;
a breath VOC analyzer in communication with the breath detector apparatus, the breath VOC analyzer operable to automatically determine a level of the VOC;
a hardware computer processor configured to compare the level of the VOC to a predetermined threshold level of the VOC, to output a disease signal if the level of the VOC is less than the threshold level of the VOC; and
a display in communication with the hardware computer processor, the display operable to provide an aural or visual alert of a disease indication.

10. The apparatus of claim 9, wherein the VOC is 1-propanol.

11. The apparatus of claim 9, wherein the breath VOC analyzer comprises an electronic nose configured to detect the VOC.

12. The apparatus of claim 11, further comprising a medical ventilator, wherein the electronic nose is disposed to directly sample exhaled breath from an exhalation tube.

13. The apparatus of claim 9, wherein the breath VOC analyzer comprises a gas chromatography-mass spectrometry (GC-MS) analyzer.

14. The apparatus of claim 13, wherein the breath detector apparatus comprises a bag disposed to capture the exhaled breath and the VOC.

15. The apparatus of claim 13, wherein the breath detector apparatus comprises a sorbent tube and a pump, wherein the pump is disposed to draw a predetermined sample volume of the exhaled breath through the sorbent tube, the sorbent tube further arranged to retain the VOC.
